(19) United States
(12) Patent Application Publication
(10) Pub. No.: US 2016/0074674 A1
(43) Pub. Date: Mar. 17, 2016

(54) COMBINED RESPIRATION AND CARDIAC GATING FOR RADIOTHERAPY USING ELECTRICAL IMPEDANCE TECHNOLOGY

(71) Applicant: BRITISH COLUMBIA CANCER AGENCY BRANCH, Vancouver (CA)

(72) Inventors: Kirpal KOHLI, Surrey (CA); Steven THOMAS, White Rock (CA); Ash M. PARAMESWARAN, Coquitlam (CA); Feng-yu LIU, Vancouver (CA)

(21) Appl. No.: 14/783,705

(22) PCT Filed: Feb. 27, 2014
(86) PCT No.: PCT/CA2014/050139

§ 371 Date: Oct. 9, 2015

Related U.S. Application Data
(60) Provisional application No. 61/810,927, filed on Apr. 11, 2013.

(51) Int. Cl. A61N 5/10; A61B 5/0402; A61B 5/00; A61B 5/0205

(52) U.S. Cl. CPC A61N 5/1068; A61B 5/0205; A61B 5/0402; A61B 5/4836; A61B 5/053

(57) ABSTRACT

A gating system uses measurements of electrical impedance of a subject to provide simultaneous gating for respiratory and cardiac motion. The gating is based on the change in bio impedance that occurs across trans-thoracic electrodes during breathing and cardiac motion. These quantities can be measured non-invasively in real time by transmitting a known low-amplitude and low-frequency current and measuring voltage drop across electrodes attached to the thorax. The gating signals may control delivery of radiation by a radiotherapy device or an imaging device.

### CROSS-REFERENCE TO RELATED APPLICATION

This application claims priority from U.S. patent application No. 61/810,927 filed on Apr. 11 2013 and entitled: SIMULTANEOUS LUNG AND CARDIAC GATED RADIOTHERAPY USING ELECTRICAL IMPEDANCE TECHNOLOGY which is hereby incorporated herein by reference for all purposes.

### TECHNICAL FIELD

This invention relates to radio therapy and in particular to gated radiotherapy. Embodiments provide apparatus and methods for use in gated radiotherapy.

### BACKGROUND

Radiotherapy involves delivery of radiation to tissues. For example, radiotherapy may involve delivering radiation to a tumor. Various types of radiation delivered from different radiation sources may be used in radiotherapy. For example, some radiotherapy involves delivery of high-energy X-rays to target tissues (typically megavoltage X-rays). Such X-rays may, for example, be generated by a linear accelerator (LINAC). Other types of radiotherapy may deliver particles such as beams of electrons, protons, positrons or neutrons to target tissues.

Modern radiotherapy delivery systems are capable of delivering radiation with significant precision. High precision targeting of target tissues can allow adjacent tissues to be spared. Reducing radiation dose to normal tissues can reduce the side effects of radiation treatment.

One problem that can interfere with the targeted delivery of radiation is that target tissues may move. For example, a target tissue may move with cardiac and/or respiratory cycles. Motions resulting from respiration and cardiac cycles are a particular issue when delivering thoracic, upper abdominal or breast radiotherapy. For example, tissue motion may be a factor when treating tumors of the lung, breast, pancreas and liver. This problem may be addressed by gating the radiotherapy such that radiation is delivered only during selected phases of the cardiac and/or respiratory cycles.

Respiratory gating of radiation therapy involves limiting delivery of radiation to optimum parts of the respiratory cycle. The position and width of the gate within a respiratory cycle may be determined by monitoring the patient's respiratory motion, using either an external respiration signal or internal fiducial markers. Literature describing respiration gated radiotherapies includes:
Kubo H D and Hill B C 1996 Respiration gated radiotherapy treatment: a technical study Phys. Med. Biol. 41 83-91;
Ramsey C R, Scaperoth D, and Arwood D 2000 Clinical experience with a commercial respiratory gating system Int. J. Radiat. Oncol. Biol. Phys. 48(3) 164-165;
Seiler P G, Blattmann H, Kirsch S, Muench R K, and Schilling C 2000 A novel tracking technique for the continuous precise measurement of tumor positions in conformal radiotherapy Phys. Med. Biol. 45 103-110;
Tang X, Sharp G C and Jiang S B 2007 Fluoroscopic tracking of multiple implanted fiducial markers using multiple object tracking Phys Med Biol. 52(14) 4081-4098;
Shirato H et al 2003 Feasibility of insertion/implantation of 2.0-mm-diameter gold internal fiducial markers for precise setup and real-time tumor tracking in radiotherapy Int J Radiat Oncol Biol Phys. 56(1) 240-247; and
Keall P J et al 2006 The management of respiratory motion in radiation oncology report of AAPM Task Group 76 Med Phys 33(10) 3874-900.

One example of a commercially available respiratory gating system is the Real-time Position Management™ (RPM) system available from Varian Medical Systems of Palo Alto, Calif., USA. In this system an external marker device is placed on the abdomen between the xyphoid process and the umbilicus. An infrared camera tracks the motion of the marker, and that motion generates a surrogate for the respiratory cycle.

Respiratory position monitoring of the type provided by the RPM system has been extremely valuable to radiation oncology but is not ideal for all patients and suffers from some shortcomings. The marker device is difficult to position in patients with certain body habitus and is poorly mobile in certain patients who do not breathe with their diaphragm while patients with poor lung function have little chest/abdominal wall excursion so the marker device does not move and a respiratory tracing cannot be obtained. There may be an inherent lag between motion of internal anatomy and motion of the external marking device.

The use of an external marker to monitor respiration is also complicated because patients with emphysema can exhibit paradoxical diaphragm motion (both as a single structure and with respect to the ventral rib cage). This is described in Iwasawa T, Yoshiike Y, Saito K, Kagei S, Gotoh T, and Matsubara S 2000 Paradoxical motion of the hemidiaphragm in patients with emphysema J Thorac Imaging 15(3)191-195. As the population of lung cancer patients presenting for radiotherapy contains many patients with compromised pulmonary function, concerns about the use of the diaphragm as a surrogate indicator of lung tumor motion are extremely relevant.

Seiler P G, Blattmann H, Kirsch S, Muench R K, and Schilling C 2000 A novel tracking technique for the continuous precise measurement of tumor positions in conformal radiotherapy Phys. Med. Biol. 45 103-110 describes an internal marker based gating system comprising a miniature, implantable powered radiofrequency (RF) coil that can be tracked electromagnetically in three dimensions from outside the patient. Keall P J et al 2006 The management of respiratory motion in radiation oncology report of AAPM Task Group 76 Med Phys 33(10) 3874-900 describes the performance of a wireless RF seed tracking system for tumor localization. Even though this system is considered to be accurate, it involves an invasive procedure and there are minor risks associated with this, including pain, bleeding, and infection. In addition, tracking a few localized internal markers does not comprehensively account for the motion of the many normal structures adjacent to tumors.

Some attempts have been made to generate gating signals by tracking a few localized internal markers. Such markers do not provide comprehensive accounting of motion of normal structures adjacent to tumors. Beacon transponders are safe for magnetic resonance imaging (MRI); however, when imaged with MRI, a local image artifact will appear in tissue adjacent to the implanted transponders. This MRI susceptibility artifact can extend up to 2 cm from the transponder locations.

Weinberger et al., U.S. Pat. No. 5,764,723 discloses apparatus that includes a radiation applicator arranged to apply radiation to a patient in response to a trigger signal. The trigger signal is generated by a controller in response to outputs from an electrocardiograph and a respiratory monitor.

Koivumaki T, Vauhkonen M, Kuikka J T, and Hakulinen M A 2011 Optimizing bio-impedance measurement configuration for dual-gated nuclear medicine imaging: a sensitivity study Med. Biol. Eng. Comput. 49 783-791 describes the use of bio-impedance signals for gating in nuclear medicine imaging. The disclosed methods involved filtering out a Direct Current (DC) component of the sampled signal in order to remove baseline fluctuations of the respiratory signal. However, in events such as momentary breath holding, the filtered sampled signal cannot correctly represent the position of the chest cavity, which will result in error if the signal is to be used for gating the actual treatment.

Koivumaki T, Vauhkonen M, Kuikka J T, and Hakulinen M A 2012 Bio-impedance-based measurement method for simultaneous acquisition of respiratory and cardiac gating signals Physiological Measurement 33 1323-1334 describes sampling a raw bio-impedance based electrical signal and then extracting respiratory and cardiac components by digital signal processing using software. The digital processing added significant delay.

An effective gating system may allow radiation fields to be more tightly focused on target tissues (i.e. have smaller internal target volume (ITV) margins) without compromising coverage of the target volume. This will allow improved sparing of normal tissues such as the heart, lungs and mediasintal structures such as the proximal bronchial tree, and central vasculature.

The inventors have recognized a need for a gating system that is effective, operates accurately in real time to track stages of the respiratory and/or cardiac cycle and is simple to apply.

### SUMMARY

This invention has a number of aspects. These aspects include: gating systems for use in radiotherapy and/or imaging (e.g. CT imaging); radiotherapy apparatus that includes a gating system; imaging apparatus (e.g. a CT imaging system) that includes a gating system; methods for providing gating signals; methods for delivering radiotherapy; and imaging methods.

One example aspect provides apparatus for gating delivery of radiation to a subject. The apparatus comprises a signal generator having first and second outputs respectively connectable to first and second electrodes. The signal generator is operative to apply an electrical sensing signal between the first and second electrodes. The apparatus also comprises first and second monitoring circuits. The first monitoring circuit is configured to monitor characteristics of the electrical sensing signal to yield a first output signal representative of an electrical impedance between the first and second electrodes. The second monitoring circuit has first and second inputs connectable to third and fourth electrodes and is configured to monitor an electrical potential between the third and fourth electrodes to yield a second output signal. The second monitoring circuit comprises an analog filter having a bandpass filter characteristic with a passband including frequencies in the range of 1-2 Hz. The apparatus also comprises a gating circuit connected to process the first and second output signals to yield a gating signal. The gating signal may be applied to enable and/or inhibit delivery of radiation by a radiation-emitting apparatus such as a radiation treatment machine (e.g. a linear accelerator), an imaging machine (e.g. an X-ray machine, computed tomography (CT) imaging system or the like).

In some embodiments the electrical sensing signal has a frequency exceeding 1 kHz (e.g. 50 kHz±15 kHz in some embodiments) and the first and second monitoring circuits each comprise an analog filter tuned to pass the frequency of the electrical sensing signal.

In some embodiments the first monitoring circuit comprises a first signal amplitude detector and a first difference circuit connected to subtract a first DC offset from an output of the first signal amplitude detector upstream from the first analog filter. A control circuit may be connected to control a magnitude of the subtracted DC offset. The difference circuit may, for example, comprise a difference amplifier. The control circuit may, for example, comprise a digital to analog converter having an output connected to set a voltage applied to one input of the difference amplifier.

In some embodiments the control circuit comprises a programmable processor configured by software to monitor a DC component of a signal output by the difference amplifier and to dynamically vary the set voltage to reduce the DC component of the signal output by the difference amplifier to be below a threshold.

In some embodiments the second monitoring circuit comprises a second signal amplitude detector and a second difference circuit connected to subtract a second DC offset from an output of the second signal amplitude detector upstream from the second analog filter.

In some embodiments the first monitoring circuit comprises an analog filter having a low pass or bandpass filter characteristic downstream from the second difference circuit. The analog filter may pass frequencies characteristic of respiration.

Gating may be based on one or more of a number of criteria. In some embodiments the gating circuit is based in part on a DC component of a signal in the first monitoring circuit. For example, in some embodiments the gating circuit is configured to monitor a rate of change of a DC component of the output of the first signal amplitude detector and to set the gating signal to inhibit radiation delivery if the rate of change meets or exceeds a threshold. In some embodiments the gating circuit is configured to monitor a difference between a DC component of the output of the first signal amplitude detector at a first time and a present time and to set the gating signal to inhibit radiation delivery if the difference meets or exceeds a threshold.

In some embodiments the gating circuit is configured to monitor a phase of the first output signal and a phase of the second output signal and to set the gating signal to inhibit radiation delivery unless the phase of the first output signal and the phase of the second output signal each satisfy a predetermined criterion. In some embodiments the gating circuit is configured to monitor an amplitude, frequency or amplitude and frequency of an AC component of the first output signal and to set the gating signal to inhibit radiation delivery based at least in part on values of the amplitude, frequency or amplitude and frequency of the AC component. In some embodiments the gating circuit is configured to periodically sample a DC component of the output of the first signal amplitude detector and to set the gating signal to inhibit delivery of radiation if more than a threshold number of the samples in a current time window deviate from a predefined range. The predefined range may be, for example, a range around an average or median value of the samples. In some embodiments the gating circuit is configured to set the gating signal to inhibit delivery of radiation if a rate of change of the frequency or amplitude of an AC component of the first output signal exceeds a threshold rate. In some embodiments the gating circuit is configured to set the gating signal to inhibit delivery of radiation if a frequency or amplitude of an AC component of the first output signal is outside of a predetermined range.

In some embodiments the apparatus comprises a differentiating amplifier connected to output a rate of change of a frequency and/or amplitude of an AC component of the first output signal.

The gating circuit may, for example, comprise one or more analog-to-digital converters connected to sample the first and second output signals and a programmable processor configured by software to generate the gating signal based at least in part on the sampled first and second output signals. In some embodiments the programmed processor is replaced by and/or augmented by logic circuits and/or configurable logic devices.

In some embodiments the apparatus comprises an ECG circuit connected to process the potential different at the inputs of the second monitoring circuit to yield an ECG output signal. For example, the ECG circuit may comprise a filter circuit configured to detect and amplify frequencies in the range of about 0.8 Hz to about 100 Hz and to suppress other frequencies. In some embodiments the gating circuit is connected to receive the ECG output signal and is configured to generate the gating signal based in part on the ECG signal. For example, the gating circuit may be configured to inhibit delivery of radiation unless the ECG output signal and the second output signal each satisfy predetermined criteria.

Another example aspect of the invention provides methods for generating gating signals for gating delivery of radiation to a subject. The methods comprise applying an electrical sensing signal between first and second electrodes in contact with a subject; measuring an impedance between the first and second electrodes to produce an impedance signal; measuring a voltage between third and fourth electrodes in contact with the subject to produce a voltage signal and processing the voltage signal to determine an amplitude of the voltage signal; filtering the impedance signal in the analog domain to remove signal components with frequencies above a first threshold frequency to produce a first output signal; filtering the processed voltage signal in the analog domain to remove signal components outside of a frequency band, the frequency band including frequencies in the range of 1-2 Hz, to produce a second output signal; and processing the first and second output signals to generate a gating signal. In some embodiments, before filtering the impedance signal, the method measures the amplitude of the impedance signal; and subtracts a first DC offset from the amplitude of the impedance signal. Some such methods comprise adjusting the first DC offset to maintain the amplitude of the impedance signal below a threshold. This adjustment may, for example, be performed by a feedback control circuit. Some embodiments comprise, before filtering the voltage signal subtracting a second DC offset from the amplitude of the voltage signal.

The electrodes may, for example, be located 46. with the first pair of electrodes along mid-axillary line on both the right and left sides of the subject's chest, one electrode of the second pair of electrodes is located at the level of the subject's xiphoid and a second electrode of the second pair of electrodes located a short distance (e.g 2 cm) lateral of the one electrode on the left side.

In some embodiments, processing the first and second output signals to generate a gating signal comprises: monitoring a rate of change of a DC component of the impedance signal; and generating a gating signal that inhibits radiation delivery if the rate of change meets or exceeds a threshold.

Processing the first and second output signals to generate a gating signal may comprise one or more of:
monitoring a difference between a DC component of the impedance signal at a first time and a present time; and generating a gating signal that inhibits radiation delivery if the difference meets or exceeds a threshold.
monitoring a phase of the first output signal and a phase of the second output signal; and generating a gating signal that inhibits radiation delivery unless the phase of the first output signal and the phase of the second output signal each satisfy a corresponding predetermined criterion.
monitoring an amplitude, frequency or amplitude and frequency of an AC component of the first output signal; and generating a gating signal that inhibits radiation delivery based at least in part on values of the amplitude, frequency or amplitude and frequency of the AC component.
periodically sampling a DC component of the impedance signal; and generating a gating signal that inhibits radiation delivery if more than a threshold number of the samples in a current time window deviate from a predefined range.
generating a gating signal that inhibits radiation delivery if a rate of change of a frequency or amplitude of an AC component of the first output signal exceeds a threshold rate.
generating a gating signal that inhibits radiation delivery if a frequency or amplitude of an AC component of the first output signal goes outside a predetermined range.
   Some embodiments comprise sampling the first and second output signals (e.g. with one or more analog to digital converters) and generating the gating signal based at least in part on the sampled first and second output signals.

Another example embodiment provides apparatus for gating delivery of radiation to a subject. The apparatus comprises a first pair of electrodes for placing on either side of a subject's torso; a second pair of electrodes for placing on the subject's torso in a vicinity of the subject's heart; a first impedance-sensing circuit configured to monitor a first bioimpedance between the first pair of electrodes and to generate a respiration signal indicative of a phase of the subject's respiration cycle from the monitored first bioimpedance; a second impedance-sensing circuit connected to monitor a potential difference between the second pair of electrodes and configured to monitor a second bioimpedance between the second pair of electrodes and to generate a cardiac signal indicative of a phase of the subject's cardiac cycle from the monitored second bioimpedance; an ECG circuit configured to generate a ECG signal from the potential difference between the second pair of electrodes; and a gating circuit connected to receive the cardiac signal and the respiration signal and configured to generate a gating signal based on at least the cardiac signal and the respiration signal. Some such apparatus can provide a gating signal based on both cardiac and respiratory cycles of a subject and an ECG signal using only four electrodes.

In some embodiments the gating circuit is configured to generate the gating signal based in part on the ECG signal.

Another example aspect provides apparatus for gating delivery of radiation to a subject. The apparatus comprises a first pair of electrodes for placing on either side of a subject's torso; a second pair of electrodes for placing on the subject's torso in a vicinity of the subject's heart; a first impedance-sensing circuit configured to monitor a first bioimpedance between the first pair of electrodes and to generate a respiration signal indicative of a phase of the subject's respiration cycle from the monitored first bioimpedance; a second impedance-sensing circuit connected to monitor a potential difference between the second pair of electrodes and configured to monitor a second bioimpedance between the second pair of electrodes and to generate a cardiac signal indicative of a phase of the subject's cardiac cycle from the monitored second bioimpedance; and a gating circuit connected to receive the cardiac signal and the respiration signal and configured to generate a gating signal based on at least the cardiac signal and the respiration signal. The first impedance sensing circuit may be configured to subtract a DC offset from the monitored first bioimpedance. The gating circuit may be connected to receive a signal indicative of a magnitude of the DC offset and to generate a gating signal based at least in part on the magnitude of the DC offset.

In some embodiments the gating circuit is configured to monitor a rate of change of the DC offset and to set the gating signal to inhibit radiation delivery if the rate of change meets or exceeds a threshold. In some embodiments the gating circuit is configured to store a value of the DC offset at a first time and to compute a difference between the DC offset and the stored value of the DC offset and to set the gating signal to inhibit radiation delivery if the difference meets or exceeds a threshold.

Another example aspect provides methods for creating signals for gating delivery of radiation to a subject. The methods comprise monitoring a first bioimpedance between the first pair of electrodes on either side of a subject's torso and generating a respiration signal indicative of a phase of the subject's respiration cycle from the monitored first bioimpedance; monitoring a second bioimpedance between a second pair of electrodes on the subject's torso in a vicinity of the subject's heart based on a potential difference between the second pair of electrodes and generating a cardiac signal indicative of a phase of the subject's cardiac cycle from the monitored second bioimpedance; subtracting a DC offset from the monitored first bioimpedance; and generating a gating signal based at least in part on the magnitude of the DC offset.

In an example embodiment the method comprises one or more of:
monitoring a rate of change of the DC offset and setting the gating signal to inhibit radiation delivery if the rate of change meets or exceeds a threshold.
storing a value of the DC offset at a first time; computing a difference between the DC offset and the stored value of the DC offset and setting the gating signal to inhibit radiation delivery if the difference meets or exceeds a threshold.
monitoring a phase of the cardiac signal and a phase of the respiration signal and setting the gating signal to inhibit radiation delivery unless the phase of the cardiac signal and the phase of the respiration signal each satisfy a predetermined criterion.
monitoring an amplitude, frequency or amplitude and frequency of an AC component of the respiration signal and setting the gating signal to inhibit radiation delivery based at least in part on values of the amplitude, frequency or amplitude and frequency of the AC component.
periodically sampling the DC offset and setting the gating signal to inhibit delivery of radiation if more than a threshold number of the samples of the DC offset in a current time window deviate from a predefined range.

Apparatus as described herein may be provided as stand-alone apparatus but may also be integrated with other apparatus such as a radiation delivery apparatus (e.g. a linear accelerator or other therapeutic radiation delivery system, an imaging system etc.).

Further aspects and example embodiments are illustrated in the accompanying drawings and/or described in the following description.

### BRIEF DESCRIPTION OF THE DRAWINGS

The accompanying drawings illustrate non-limiting example embodiments of the invention.

FIG. 1 is a block diagram illustrating radiotherapy and imaging apparatus according to an example embodiment.

FIGS. 1A and 1B show example arrangements of electrodes for acquisition of bioimpedance measurements from which respiratory and cardiac signals may be derived.

FIG. 2 shows an example bioimpedance signal from which a respiratory gating signal may be obtained.

FIG. 3 is a block diagram of a bioimpedance monitoring apparatus.

FIG. 4 is a schematic diagram for a prototype example gating circuit.

FIG. 5 shows software algorithms.

FIG. 6 shows dataflow in an example gating system.

FIG. 7 is a plot showing impedance signals as a function of time for several breathing patterns.

FIGS. 7A to 7E show real time respiratory and cardiac signals for various breathing modes.

FIGS. 8A to 8D show real time respiratory, cardiac and ECG signals acquired simultaneously.

### DESCRIPTION

Throughout the following description specific details are set forth in order to provide a more thorough understanding to persons skilled in the art. However, well known elements may not have been shown or described in detail to avoid unnecessarily obscuring the disclosure. The following description of examples of the technology is not intended to be exhaustive or to limit the system to the precise forms of any example embodiment. Accordingly, the description and drawings are to be regarded in an illustrative, rather than a restrictive, sense.

Embodiments of the invention use bioimpedance measurements to monitor cardiac and/or respiratory cycles. The same apparatus may monitor both cardiac and respiratory cycles based on measurement of bioimpedance. Bioimpedance signals are processed to provide real-time indications of the stages of cardiac and/or respiratory cycles. This information is then applied to generate a gating signal that may control a radiotherapy device and/or an imaging system to irradiate a target volume only a selected stages of the cardiac and/or respiratory cycles. The radiotherapy device may, for example, comprise a linear accelerator.

A bioimpedance signal can be used to evaluate the phase of respiratory and/or cardiac signals because the bioimpedance of a subject's torso is different at different stages of the respiratory and cardiac cycles. In humans, an inspiration maneuver from residual volume to total lung capacity results in a regional bio impedance change of 300% (see: Barber, D. C. A review of image reconstruction techniques for electrical impedance tomography. Medical Physics, 1989; 16(2): 162-169; and Faes, T. J. C., H. A. van der Meij, J. C. de Munck, and R. M. Heethaar. The electric resistivity of human tissues (100 Hz-10 MHz): a meta-analysis of review studies. Physiology, 1999; 20(4): R1). Cardiac activity and perfusion also cause a change in thoracic bio impedance, from diastole to systole, in the range of 3% as described in Visser, K. R. Electric properties of flowing blood and impedance cardiography. Annals of Biomedical Engineering, 1989; 17: 463-473.

Bioimpedance may be monitored to observe the change ΔZ in the trans-thoracic bio impedance, Z, that occurs during breathing and cardiac motion. The bio impedance (Z) may be defined as an instantaneous ratio of voltage (V) and current (I) by Z=V/I. ΔZ(t) is the change in bioimpedance as a function of time, t.

Bioimpedance can be measured non-invasively in real time, for example by transmitting a known current (I) through the tissues of a subject and measuring the resulting potential difference between electrodes attached to the subject's thorax. The current I may be suitably low. The current may comprise an alternating current having a suitable frequency as described herein.

An optimal range of frequencies for electrical signals for studying body tissues is the range between 1 kHz and 100 kHz. In general it is desirable to avoid frequencies that are harmonics of the local power line frequency (60 Hz in North America). Some studies have reported that using a signal too close to the 10^{th} harmonic of the power line frequency can significantly decrease the safe current limit. On the other hand, high-frequency signals can be more susceptible to interference by ambient noise. In some embodiments the frequency has a frequency less than about 100 kHz. The selected frequency is preferably significantly greater than the frequencies of the respiratory and cardiac cycles (e.g. significantly greater than 5 Hz). In an example embodiment a carrier frequency of approximately 50 kHz was used.

The frequency of the signal used to monitor bioimpedance may be selected to facilitate detection of the signal and to avoid interference from electrical noise. Some embodiments use a signal having a frequency in the range of about 50 kHz to about 100 kHz. This range has been identified as having reduced interference from organic tissues and ambient high-frequency signals (see Davidson, K. G., A. D. Bersten, T. E. Nicholas, P. R. Ravenscroft, and I. R. Doyle. Measurement of tidal volume by using transthoracic impedance variations in rats. Journal of Applied Physiology, 1999; 86(2): 759-766; and, Marinova I. and V. Mateev. Determination of electromagnetic properties of human tissue. World Academy of Science, Engineering and Technology 42, 2010).

The amplitude of the signal used to monitor bioimpedance is limited to be medically safe and also not distracting to the subject. Any device that directly applies electrical currents to a human must be carefully evaluated in terms of both current and frequency. For a 70 kg human the minimum current of threshold perception for men and minimum threshold let-go current for women is 6 mA at 60 Hz (Olson W H 2008). According to current guidelines, safe current limits for electromedical apparatus are constant from DC to 1 kilohertz. Above one kHz the limit is increased proportionally to a maximum value of 100 times at 100 KHz. Therefore, at a frequency of 50 KHz the safe limit current will be 50 times the safe limit current at DC to 1 kilohertz.

In an example embodiment the signal used to monitor bioimpedance is current-limited to <1 mA. This current is so small that it cannot be felt by normal human subjects upon application. This amount of current is well within the safe current limit but is high enough facilitate detection and analysis of bioimpedance signals. In some embodiments, electrical power for circuits connected to electrodes on a patient are provided by batteries for safety reasons. In other embodiments, power is supplied from mains electricity by way of circuits that provide failsafe control over current and voltage in accordance with accepted design practices for medical electronic equipment.

As described herein, both respiratory and cardiac functions can be monitored using the same bio impedance measurements. Consequently monitoring bioimpedance signals may be applied to generate gating signals responsive to the phases of both respiratory and cardiac cycles in a non-invasive manner using a single sensor or device.

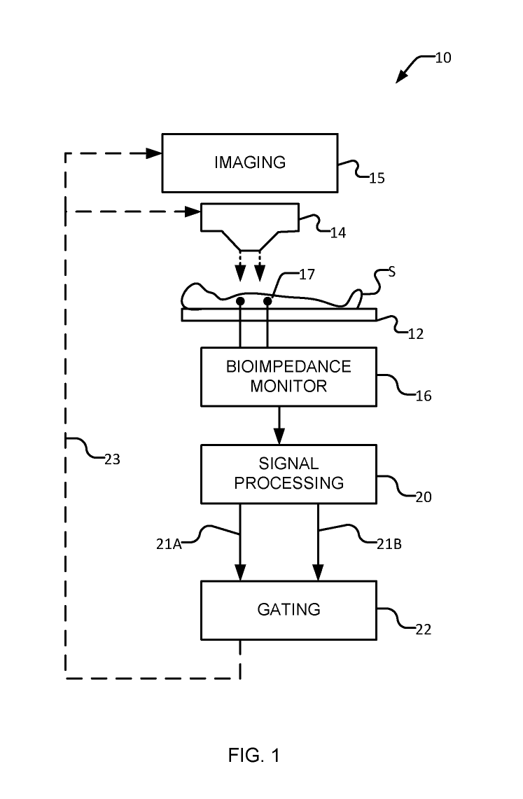

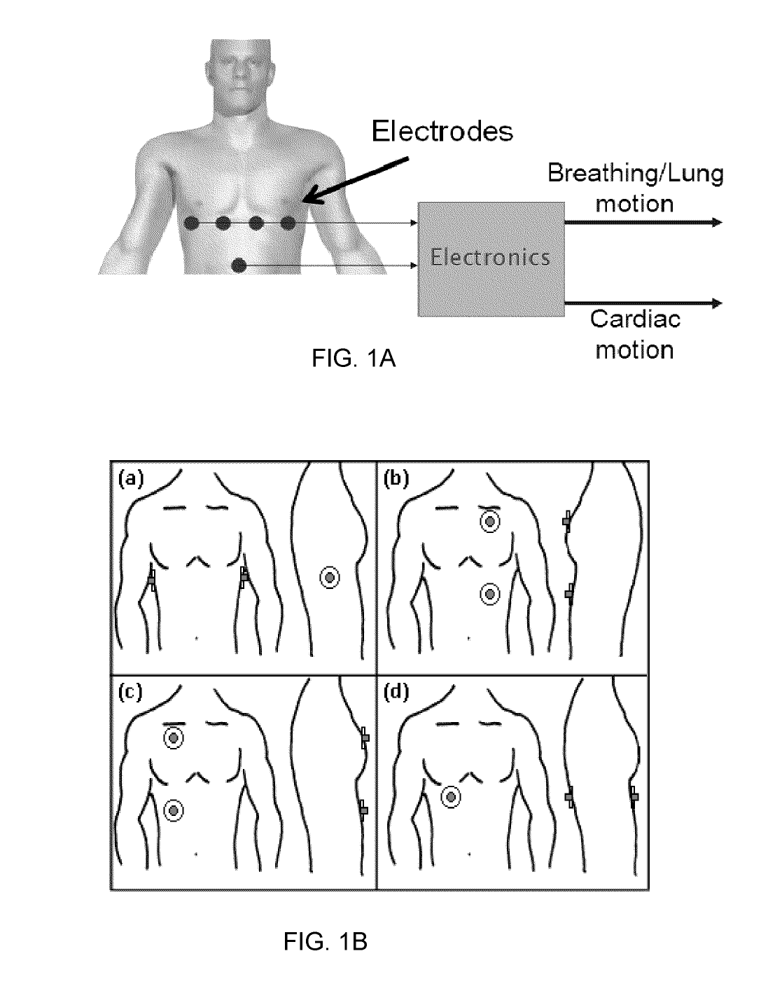

FIG. 1 is a block diagram illustrating radiotherapy apparatus 10 according to an example embodiment of the invention. Radiotherapy apparatus 10 comprises a patient support 12 such as a table supporting a subject S. A radiation delivery apparatus 14 such as a linear accelerator is arranged to deliver radiation to a target volume within subject S. The target volume may comprise, for example a tumour such as a lung tumour.

A bioimpedance monitor 16 has electrodes 17 in contact with the subject S. Electrodes 17 may, for example, comprise one or more first electrodes disposed on one side of the subject's thorax and one or more electrodes disposed on an opposing side of the subject's thorax so as to measure a trans-thoracic bioimpedance. In some embodiments, electrodes 17 comprise a pair of current delivery electrodes 17A and two or more potential-sensing electrodes 17B. For example, the first electrodes and second electrodes may comprise a set of current delivery electrodes 17A and a set of potential-sensing electrodes 17B. Potential-sensing electrodes 17B optionally provide higher-impedance coupling to the subject than current delivery electrodes 17A.

Bioimpedance monitor 16 generates a bioimpedance signal 18 which is processed by processing circuits 20 to yield a respiration signal 21A and a cardiac signal 21B. Respiration signal 21A and/or cardiac signal 21B are processed by a gating circuit 22 to yield a gating signal 23 that is applied to control radiation delivery apparatus 14. Gating signal 23 inhibits delivery of radiation by radiation delivery apparatus 14 except during a selected phase or phases of respiration signal 21A and/or cardiac signal 21B.

Gating signal 23 is also delivered to an imaging system 15 (e.g. an X-ray imaging system, an ultrasound imaging system, a computed tomography (CT) imaging system, a positron emission tomography (PET) imaging system, a magnetic resonance imaging (MRI) imaging system or any combination thereof) that may be used for imaging during or in preparation for radiation treatment. Imaging system 15 may be gated according to gating signal 23 so as to obtain images of anatomical structures in the positions that those anatomical structures will have when being irradiated with radiation from radiation delivery apparatus 14.

Bioimpedance monitor 16, signal processing 20 and gating system 22 are not necessarily separate but may be combined with one another in any suitable manner. In some advantageous embodiments, signal processing 20 is performed entirely in the analog domain.

Bioimpedance may be measured non-invasively in real time by applying electrodes to a subject, applying a potential difference between the electrodes and measuring a resulting electrical current passed between the electrodes. In some applications, electrodes are reproducibly placed at the level of a tumor or other target volume on marked locations on the patient such that the same electrode positions can be used to obtain bioimpedance signals for each of a plurality of treatments.

The electrodes may be of any suitable type. For example, Ag—AgCl disposable electrodes may be used. Radiolucent electrodes (for example of the type available from Covidien™) may also be used. Radiolucent electrodes tend to be more suitable in a radiotherapy environment as these electrodes do not create any image artifacts.

Bio-impedance changes due to cardiac motion can be simultaneously measured using the same carrier signal employed for respiratory monitoring. In some embodiments this is done by placing a second pair of electrodes on the thorax in close proximity of the heart. This second pair of electrodes may, for example, include one electrode placed along the sternum, at the level of 6th sternocostal junction, and a second electrode placed 4 cm lateral to the first electrode on the left side.

In some embodiments four electrodes are used, one pair of electrodes is used to inject an electrical current into a subject and another pair of electrodes is used to monitor a potential difference in the vicinity of the subject's heart. The locations of the first and second pairs of electrodes may be chosen so that changes in bioimpedance between the first pair of electrodes are indicative of respiratory function and are relatively independent of cardiac function and changes in bioimpedance between the second pair of electrodes are indicative of cardiac function and are relatively independent of respiratory function. The first electrodes may be placed, for example, on either side of a subject's thorax. The current and potential difference between the first electrodes may be processed to yield a signal indicative of a phase of the subject's respiratory cycle. The second electrodes may be placed near to the subject's heart, for example, on the left side of the subject's chest.

FIG. 1A shows an example arrangement of electrodes for monitoring bioimpedance of a subject's thorax. This example arrangement has five electrodes. The fifth electrode is optional. If present it may be used as a reference electrode. In this and other embodiments which provide three or more electrodes in the vicinity of the subject's heart, differential voltage measurements may be made between a plurality of pairs of the electrodes to yield a plurality of differential voltage signals that may be processed to provide information regarding the phase of the subject's cardiac cycle.

FIG. 1B shows example positions for current-injecting electrodes. View (a) shows an example where both electrodes are approximately 2-3 cm inferior to the axillary fold in the mid-axillary line on the right and left chests. View (b) shows an example where both electrodes are located along the left mid-clavicular line, one immediately inferior to the left clavicle, the other at the level of the left costal margin. View (c) shows an example where both electrodes are along the right mid-clavicular line, one immediately inferior to the right clavicle, the other at the level of the right costal margin. View (d) shows an example where one electrode is at the level of the 5th-6th rib in the mid-clavicular line, and the other electrode is directly posterior.

The electrode arrangements of FIG. 1B were tested on healthy volunteers who were instructed to breathe at a normal rate. In each case, a voltage sensing pair of electrodes was placed in the vicinity of heart, the first electrode at the level of xiphoid and second one 2 cm lateral on the left side. Measurements were performed with the subjects in the supine position as is typical during delivery of radiotherapy.

The inventors have found in testing a prototype apparatus that an electrode placement as shown in part (a) of FIG. 1B with current-injection electrodes placed along mid-axillary line on both the right and left chest yielded bioimpedance signals with higher signal-to-noise ratios than the other electrode locations shown in parts (b), (c) and (d) of FIG. 1B. This arrangement also tends to reduce coupling of the cardiac and respiratory signals.

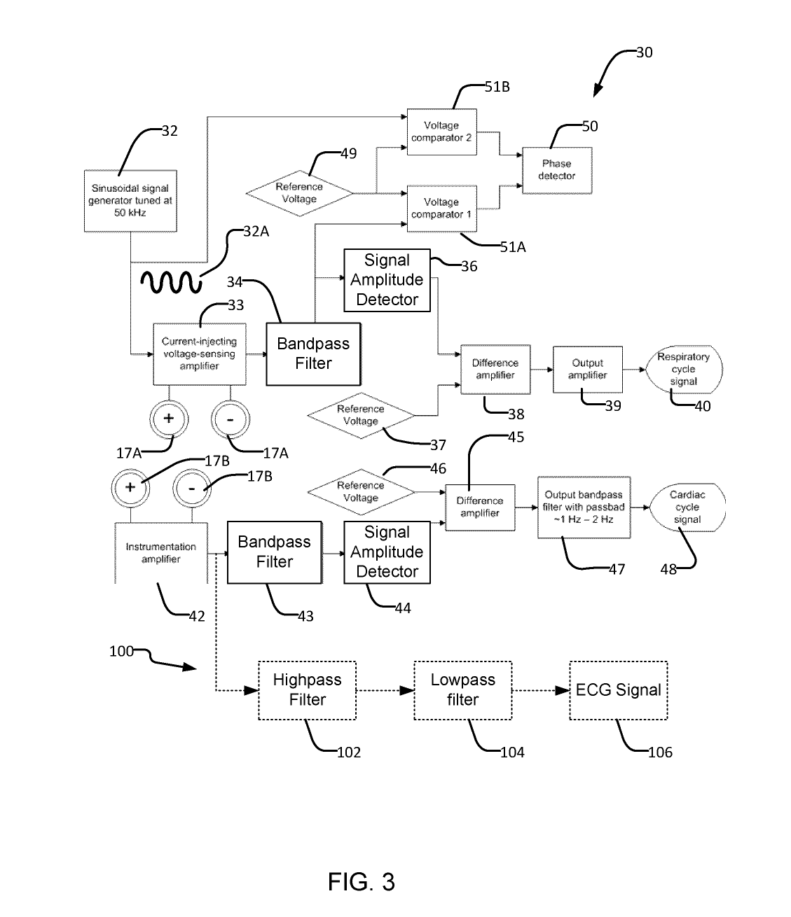

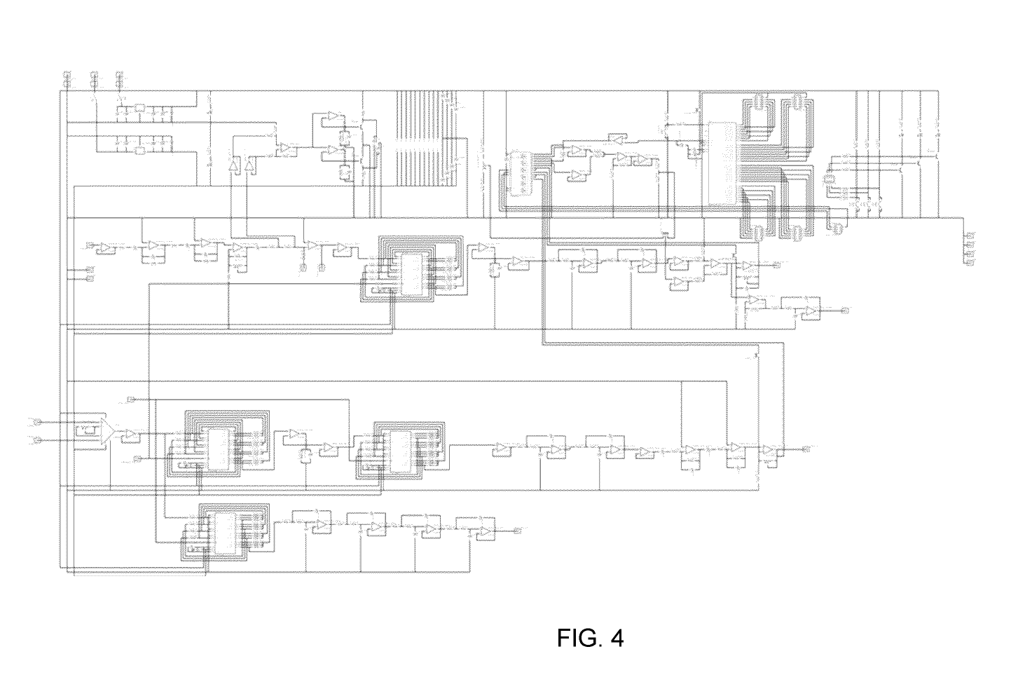

FIG. 3 is a block diagram illustrating a bioimpedance monitoring apparatus 30 according to an example embodiment. FIG. 4 is a schematic diagram illustrating a prototype bioimpedance monitoring apparatus having the general architecture illustrated in FIG. 3.

In FIG. 3, a signal generator 32 generates a signal 32A. In the illustrated embodiment the signal has a frequency of 50 kHz. Signal 32A is applied to the input of an amplifier 33 that applies an amplified version of the signal as a current signal between current delivery electrodes 17A. Amplifier 33 also senses a potential difference between electrodes 17A and provides that potential difference to a bandpass filter 34. Amplifier 33 functions as a current source where its output current is set by signal 32A. Within an operating voltage range, amplifier 33 maintains the set current independently of changes in the load (i.e. changes in the impedance between electrodes 17A).

The bandpass-filtered signal output by filter 34 is provided to signal amplitude detector 36. Amplitude detector 36 may comprise a signal envelope detector. A signal envelope detector extracts the amplitude information of a sinusoidal signal while discarding its frequency information. Amplifier 33 may also have a protective function which limits the current and voltages applied to electrodes 17A to values that have been determined to be safe.

In an example prototype embodiment the resistive component of the measured bio-impedance is monitored. Given a constant current between electrodes 17A, this resistive component is proportional to the amplitude of the carrier signal that is applied to electrodes 17A. In order to achieve high sensitivity of the correlation between the recorded signal and the breathing motion, a sharp, high-quality bandpass filter with a center frequency the same as the carrier signal frequency is used for screening out unwanted noise. In the prototype embodiment the bandpass filter was constructed using a commercially available LTC®1264 universal filter block chip (Linear Technology, Milpitas, Calif., USA). An 8th-order bandpass filter (created by cascading four 2nd-order filter blocks) amplifies signal components within the frequency band centered at the carrier frequency while largely attenuates components outside the passband frequency.

Signal amplitude detector 36 outputs a signal indicative of an amplitude of the bandpass-filtered signal from filter 34. This output signal is compared to a reference voltage 37 by difference amplifier 38 and further amplified by amplifier 39 to yield a respiratory cycle signal 40. In some embodiments the signal output from signal amplitude detector 36 is filtered, for example by a low-pass filter having a cutoff frequency above the maximum frequency expected for respiration. For example, the filter may have a cutoff frequency of 15 Hz.

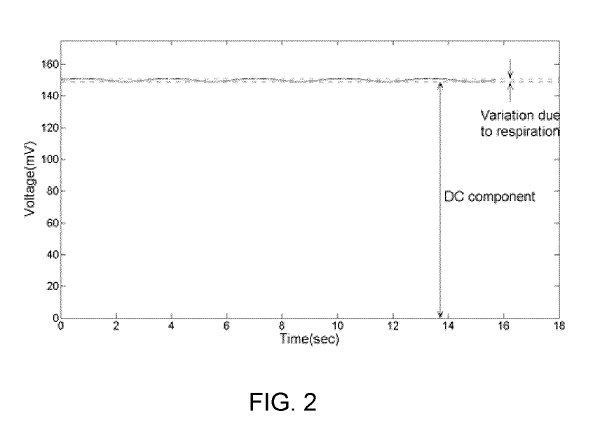

The output of signal amplitude detector 36 is typically a slowly-varying signal, of which the variations correspond to changes in bioimpedance sensed by electrode pair 17A. FIG. 2 illustrates an example output voltage of signal amplitude detector 36 as a function of time. For human subjects, the output voltage waveform of signal amplitude detector 36 is typically made up of a fluctuating component based on the respiration-induced bio-impedance changes and a DC component that is relatively large compared to the fluctuating component.

For the purpose of signal sampling and processing at the output of the detection circuit, the DC component may be removed, so that the fluctuation due to the respiratory cycle, which is the signal of interest, can be effectively isolated and amplified. One way to achieve this is to subtract all or most of the DC component. For example, in the illustrated embodiment, reference voltage 37 is set to equal or roughly equal the DC component and subtracted from the output of signal amplitude detector 36 by difference amplifier 38 to yield a signal that can be amplified by amplifier 39 to yield respiratory cycle signal 40. Subtracting reference voltage 37 to significantly reduce the DC component allows the fluctuating component to be amplified significantly without saturating amplifier 39.

In some embodiments, reference voltage 37 is controlled dynamically. For example, reference voltage 37 may be provided by a variable power supply having an electronically-controlled output voltage. In an example embodiment, reference voltage 37 is set by a digital to analog converter (DAC) under control of a processor. The processor may be configured by software instructions to monitor respiratory cycle signal 40 (either before or after amplifier 39) and to set reference voltage 37 to a value such that respiratory cycle signal 40 has a baseline of roughly 0 volts. This may be achieved, for example, by measuring a minimum value of the signal at the output of signal amplitude detector 36 over a period characteristic of respiration (e.g. 2 seconds or so). This may be done directly or calculated from a value measured downstream (e.g. at the output of amplifier 38 or 39) using information known about the gains of any intervening amplifier(s) and the current value of reference voltage 37. If this value is positive then reference voltage 37 may be increased by the measured minimum amount (or a significant fraction thereof).

Bio-impedance changes arising from cardiac motion may be simultaneously measured using the same carrier signal employed for respiratory monitoring (different signals may optionally be used). A cardiac cycle signal 48 is generated from potentials sensed by electrodes 17B which are connected to inputs of an instrumentation amplifier 42. Electrodes 17B are located close to the subject's heart. For example, one of electrodes 17B may be placed along the sternum, at the level of 6th sternocostal junction, and the other electrode 17B may be placed 4 cm lateral to the first electrode on the left side. Electrodes 17B are not called upon to source or sink any electrical current and thus cause insignificant (if any) interference with the carrier signal injected into the subject through electrodes 17A. Electrodes 17B may present a very high input impedance. In an example embodiment, instrumentation amplifier 42 is implemented using a commercially available AD620 chip (Analog Devices Norwood, Mass., USA). Instrumentation amplifier 42 measures the differential voltage between electrodes 17B while providing very large input impedance.

The output signal from amplifier 42 is filtered at bandpass filter 43 and passed to signal amplitude detector 44. Signal amplitude detector 44 outputs a signal indicative of an amplitude of the bandpass-filtered signal from filter 43. This output signal is compared to a reference voltage 46 by difference amplifier 45. The output of difference amplifier 45 is bandpass-filtered by filter 47 and optionally further amplified to yield a cardiac cycle signal 48. Filter 47 may be a low-pass filter or a bandpass filter that passes cardiac signals (which typically have frequencies in the range of about 1 Hz to about 3 Hz) and does not pass higher-frequency noise. In some embodiments, filter 47 blocks frequencies that are predominant in respiration signal 40. In an example embodiment, filter 46 comprises a high-pass filter with a cutoff frequency of below 1 Hz (e.g. 0.8 Hz) in series with a low-pass filter with a cutoff frequency of at least 5 Hz (e.g. 10 Hz).

As described above in relation to reference voltage 37, reference voltage 46 may be chosen to remove or significantly reduce a DC component of the signal output by signal amplitude detector 44. Reference voltage 46 may be dynamically adjusted in a manner analogous to that described above for reference voltage 37.

Apparatus according to some embodiments includes a controller that automatically performs an initial calibration sequence. The calibration sequence may automatically set one or more parameters such as: a magnitude of the current delivered to the subject (which must remain below safe current thresholds and is preferably as small as practical while providing useful cardiac cycle signal 48 and respiratory cycle signal 40); a frequency of the delivered current (which provides a good ratio of signal to noise in the environment in which the apparatus is being used and may also be selected to improve discrimination between cardiac and respiratory signals); reference voltages 37 and 46 which correspond to the DC components of the measured impedance signals; and gains for one or more of amplifiers 38, 39, 45, 47 (selected to yield cardiac cycle signal 48 and respiratory cycle signal 40 suitable for further processing as described herein). In an example embodiment, the controller automatically varies the carrier frequency of the current signal delivered to the subject, monitors a quality of the resulting signals and selects a frequency providing the best signal quality. The monitored quality may comprise a signal to noise ratio for example.

The cardiac cycle signal 48 and respiratory cycle signal 40 may each be digitized by an analog-to-digital converter (ADC) (in some embodiments separate ADCs sample signals 40 and 48, in other embodiments one ADC is provided to sample both signals in a time-multiplexed manner).

The system illustrated in FIG. 3 includes an optional phase detector 50 which is connected to measure a phase difference between signal 32A and the signal output by amplifier 33. In the illustrated embodiment, signal 32A and the bandpass-filtered output of amplifier 33 are each compared to a reference voltage 49 by comparators 51B and 51A to yield input signals for phase detector 50. The output of phase detector 50 is indicative of the reactive component of the bioimpedance between electrodes 17A and thus provides additional information about the physiological state of the subject. The output of a phase detector such as phase detector 50 may optionally be applied as a factor in gating radiation for radiotherapy and/or imaging. The output of a phase detector such as phase detector 50 may optionally be applied to provide outputs regarding physiological characteristics of the subject.

A difference between using bioimpedance to monitor respiration and using bioimpedance to monitor the cardiac cycle is that the cardiac cycle involves smaller motions such that the respiration signal is not generally affected significantly by the cardiac cycle. however, the cardiac cycle signal can be affected significantly by the respiratory cycle. This issue may be addressed by careful filtering of the cardiac cycle signal. To achieve high sensitivity of the correlation between the recorded signal and the breathing motion, a sharp, high-quality bandpass filter with a center frequency the same as the carrier signal frequency may be used for screening out unwanted noise.

To extract the respiratory impedance signal, filter 36 may comprise a lowpass filter with a cutoff frequency at approximately 15 Hz. Because of the locations at which electrodes 17A are placed, the variation of the signal due to cardiac activity is negligible compared to that due to respiratory activity. However, even though electrodes 17B are located close to the heart, the interference due to respiration in the signal sensed is sufficiently large to distort the shape of cardiac impedance signal. To provide a clean cardiac bio-impedance signal for gating purpose, the variation contained in the sensed signal due to respiratory motion may be removed. A sharp signal filter is used to remove the respiratory component from the cardiac signal. At rest, normal human respiratory activity usually results in a signal frequency below 0.5 Hz (30 breaths/min). Meanwhile, normal human heart rate is almost always above 1 Hz (60 beats/min). The interference due to respiratory motion may be largely removed by using a sharp highpass filter having a cutoff frequency between 0.5 Hz and 1 Hz (e.g. with a cutoff frequency of 0.8 Hz). Furthermore, a lowpass filter with a cutoff frequency of, for example, 10 Hz is used to remove high frequency noise.

In the illustrated embodiment, cardiac and respiratory signals 47 and 40 are derived from bioimpedance measurements by analog circuits (hardware). This is done prior to any signal sampling step. Such an approach has the advantage of effectively reducing the time delay caused by post-sampling data processing. This is important where a real-time gating signal is desired. The hardware may be designed to provide tracking of the cardiac and respiratory phase with little delay. For example, as compared to digital filtering, hardware filters may operate more quickly because the main factor affecting speed of the analog circuits is the propagation time of electrical signals through the circuits. However, due to the relatively low frequencies of the signals to be detected and the fact that the circuit can be compact, the propagation delay can be almost negligible. Filters may be designed so that their cut-off frequencies are not too close to the fundamental frequencies of the signals to be detected to reduce signal propagation delays.

Since there can be a wide variation from subject-to subject in bioimpedance as well as in the way that bioimpedance between certain electrodes varies with respiration and with the cardiac cycle, it is advantageous for apparatus of the general type described herein to be readily adaptable to different subjects. It is not uncommon that different individuals will have significantly different amounts of trans-thoracic body impedance and changes in respiratory-induced bioimpedance. These differences can result in different magnitudes of the DC and AC components in the output signal (e.g. the signal at the output of signal amplitude detector 36). A system as described herein may include a control subsystem that automatically adjusts reference voltages 37, 46, and/or 49 to accommodate differences between subjects.

The control subsystem may optionally also, or in the alternative, adjust gains of one or more amplifiers in the signal paths leading to respiration signal 40 and cardiac signal 48. The control system may adjust these parameters to avoid saturation of output amplifier 39 and/or difference amplifier 45 while providing a respiration signal 40 and a cardiac signal 48 from which the states of the subject's respiratory and cardiac cycles can be ascertained. In some embodiments the control system exerts closed-loop control over the values of these parameters.

In some embodiments, filters have frequency characteristics that are electronically controllable. For example, one or more of filters 34, 43, and 47 may comprise switched capacitor filters. In such embodiments the control subsystem may optionally control the passbands of one or more of the filters. For example, the control subsystem may determine a heart rate of the subject and adjust the passband of filter 47 to track the subject's heart rate. In some embodiments, once the heart rate has been determined the passband of filter 47 may be reduced to provide better rejection of respiration-related changes in bioimpedance from signal 48. As another example, the control subsystem may be configured to alter the frequency of signal 32A and to control the passbands of filters 34 and 43 to match the frequency of signal 32A. This may be done to reduce interference from extraneous signals.

In some embodiments information about the DC offsets is applied to assist in providing gating signals and/or to provide more information regarding the physiological state of the subject. For example, the DC component may change if a subject tenses up and experiences more muscle contraction. This can be detected by monitoring the DC component (either directly or by monitoring reference voltage 37 if it is set dynamically). In some embodiments, gating is temporarily inhibited during periods when a rate of change of the DC component exceeds a threshold. In some embodiments a human-perceptible signal (e.g. a visual indicator such as a lamp or a displayed indicia; and/or a sound; and/or a tactile indicator) is generated to indicate the detected change in the subject.

As another example, the subject's position and posture can affect the impedances between different pairs of the electrodes. In some embodiments these impedances are monitored for changes that may indicate that the subject has moved. For example, DC components of the impedances between one or more pairs of electrodes may be measured initially with the subject in a desired position and posture for imaging and/or radiation treatment. The initial values may be recorded in a data store such as a register, memory, or the like. These DC component(s) may be monitored over time and compared to the initial values. If the DC component(s) differ from the initial values by more than a threshold amount then gating may be set to inhibit radiation delivery. In some embodiments a human-perceptible signal is generated to indicate the detected change in the subject's position and/or posture.

As another example, the relative frequency and/or amplitude of the AC component in the respiratory signal can reflect the level of a subject's nervousness and/or the onset of a cough, sneeze, hyperventilation or other interruption in normal breathing. Gating may be inhibited, for example, if one or more of:
a rate of change of the frequency and/or amplitude of the AC component (e.g. a first time derivative of respiratory signal 40) exceeds a threshold rate; or
the frequency and/or amplitude of the AC component is outside of a predetermined range; or
the frequency and/or amplitude of the AC component differ by more than a threshold amount from a reference frequency and/or a reference amplitude respectively.
   Apparatus as described herein may optionally generate a human-perceptible signal if one or more of these events occur. In some embodiments a rate of change of the frequency and/or amplitude of the AC component is determined in the analog domain (e.g. by a differentiating amplifier). An analog output indicative of the rate of change may be compared to a threshold value by a comparator circuit and/or sampled by an ADC and compared to a threshold in the digital domain. In other embodiments the rate of change of the frequency and/or amplitude of the AC component is determined by processing in the digital domain.

As another example, changes over time in the DC component of the signal at the output of signal amplitude detector 36 may indicate momentary breath holding. Momentary breath holding may be identified by monitoring for such changes in the DC component. In an example embodiment, the magnitude of the DC component is sampled periodically, for example at a rate of a few Hz. The most-recent N samples (i.e. the samples within a moving time window) are compared to one another. If more than a threshold number of the samples deviate from a predefined range (for example a range around an average or median value of the N samples) then momentary breath holding may be occurring. Momentary breath holding can affect the configuration of the chest cavity such that the chest cavity has a different configuration than it would have in the same phase of regular breathing. Therefore, in the case of momentary breath holding the filtered sampled respiratory signal 40 may not correctly represent the position of the chest cavity, which will result in error if the signal is to be used for gating radiation. Consequently, in some embodiments, gating is inhibited in the event that momentary breath holding is detected.

Respiratory cycle signal 40 and cardiac cycle signal 48 are processed to derive a gating signal. This processing may be performed in the analog and/or in the digital domain. In some embodiments respiratory cycle signal 40 and cardiac cycle signal 48 are each sampled and the sampled signals are processed in a data processor to yield a gating signal.

In some embodiments, a gating signal is generated to allow radiation exposure for imaging and/or treatment during selected phases of the cardiac and respiratory cycles. Where such embodiments are being used, a subject may be asked to relax and breathe regularly. The system may then generate respiratory and cardiac signals as described above and output gating signals during those time periods where the phases of the cardiac and respiratory signals are both within desired ranges. In some embodiments generation of the gating signals is inhibited when anomalous events are detected (e.g. the onset of a breathing interruption such as a cough, sneeze or hyperventilation; momentary breath holding; excessive tension/nervousness; or the like).

In some embodiments, a gating signal is generated for breath hold cardiac gated treatment. In such embodiments the gating signal may be generated during those periods when the respiratory signal indicates that the subject is holding his or her breath in a steady manner and the cardiac signal indicates a that the cardiac cycle is within a desired phase range for treatment or imaging. For example, the gating signal may be inhibited unless the respiration signal remains within a narrow range of values (indicating that the subject is continuing to hold his or her breath).

Some embodiments provide a plurality of user-selectable gating modes. For example a system may provide a gating mode for use in cases where a subject is instructed to breathe regularly and another gating mode for use in cases where the subject is instructed to hold his or her breath.

A gating system as described herein may be applied to imaging as well as or instead of to radiation treatments. In some embodiments a gating system as described herein is applied to control imaging at the time of radiation treatment. By gating radiation used for imaging (e.g. X-rays) in the same manner as gating radiation used in radiation therapy, the imaging can determine what positions organs or other anatomical structures will be in during application of radiation in a radiation treatment. It has been shown that the apparent positions of intrathoracic organs obtained by a free-breathing CT scan is not representative of an average position between inhalation and exhalation (see for example, Giraud P et al 2000 Evaluation of intra thoracic organs mobility using CT gated by a spirometer Proceedings of the 19th ESTRO, Istanbul, Turkey, (PMB)). The use of respiratory or respiratory and cardiac gating during CT imaging for radiation planning can improve the positioning accuracy of tumors and normal tissues by identifying their true locations at certain phases of the respiratory and/or cardiac cycles rather than blurring their locations throughout the cycles.

A bioimpedance detection unit with the above mentioned characteristics may be combined with a computer system (e.g. one or more microcontrollers, a host computer or the like). The computer system may control operation of the bioimpedance detection unit (e.g. control the application of the carrier signal) and may process the respiratory and cardiac signals output by the bioimpedance detection unit to yield gating signals.

Gating signals may be generated based on the amplitude and/or the phase of the respiratory and/or cardiac signals. The optimum range of amplitude and/or phase to be used for gating during the treatment may be chosen to maximize dose delivery to target area with minimal dose to critical structures outside of the target area. In some applications, the ranges of amplitude and/or phase of the respiratory and/or cardiac signals for which the gating signal enables delivery of radiation corresponds to quiescent periods of the cardiac cycle. However, there may be some circumstances in which it is preferable to enable delivery of radiation during phases of the cardiac cycle when the heart is in motion.

In some embodiments, during imaging, a time of image acquisition may be recorded along with the respiration and cardiac signals for subsequent correlation of image data with respiratory and cardiac phase. Gating signals may then be synchronized with the respiration and cardiac signals during radiation treatment delivery so that radiation treatment can be delivered at appropriate times to maximize dose to a tumour (or other target area) and to minimize dose to surrounding critical structures.

In a prototype embodiment a bioimpedance detection unit incorporated a microcontroller connected to a host computer by an interface. The microcontroller was configured by software to facilitate access of the detection circuit by the host computer and also to improve the robustness of the detection mechanism. The microcontroller was programmed to issue instructions to the analog-to-digital converter, perform simple (low latency) signal conditioning on the sampled signals and relay the voltage values sampled by the A/D converter to the host computer.

In some embodiments, filters (e.g. filters 34, 43 and/or 47 in FIG. 3) are implemented using integrated circuit (IC) filters. Some such IC filters, e.g. switched capacitor filters, have frequency responses that are set by the frequency of a clock signal. In such embodiments, a microcontroller may be applied to generate clock signals for the IC filter blocks. Of course, other sources of appropriate clock signals could be provided in the alternative.

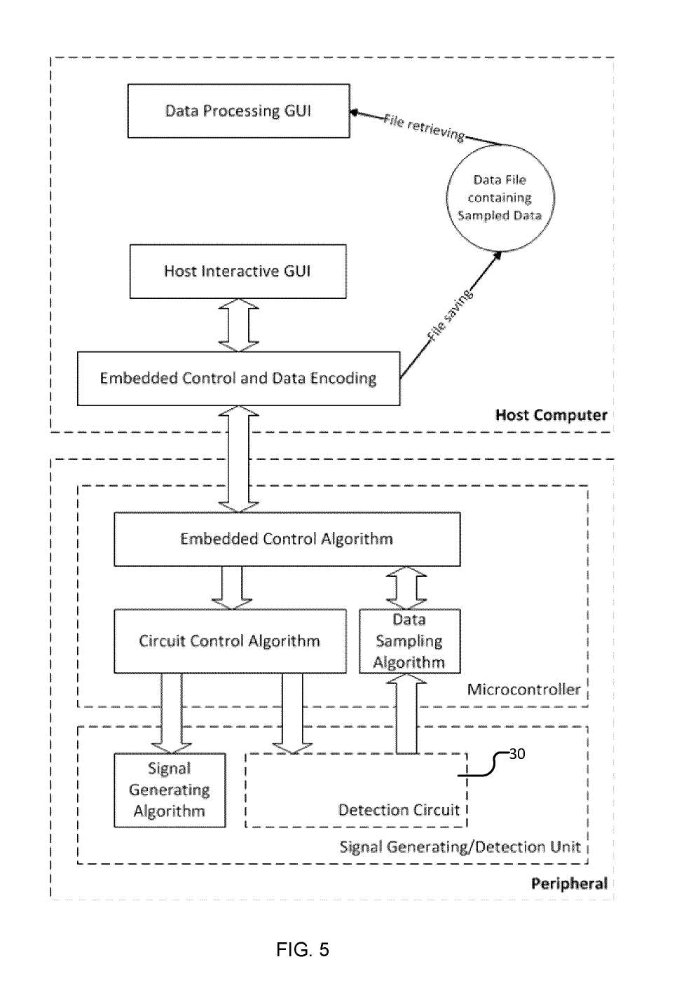

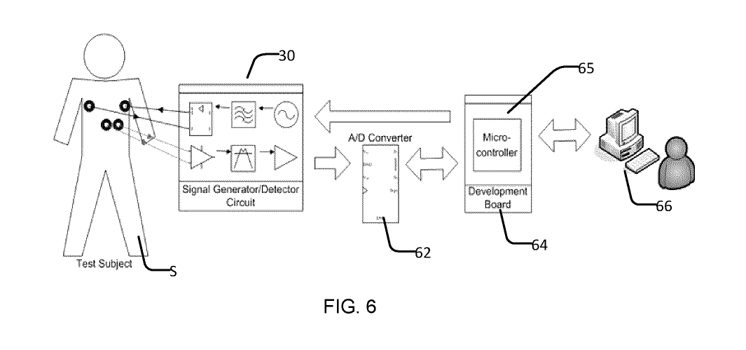

The host computer provided a graphical user interface which allowed personnel to see the respiratory and cardiac signals and to control operation of the apparatus. FIG. 5 is a block diagram illustrating the interaction of different blocks of software in an example embodiment. FIG. 6 shows an example data flow.

As illustrated in FIGS. 5 and 6, analog signals output by detection circuit 30 are sampled by an ADC 62. This may be accomplished by microcontroller 65 periodically switching on ADC 62 to sample the output voltage levels. After each conversion cycle, sampled data is transmitted to host computer 66 by microcontroller 65. This may be done using any suitable data communication protocol. In the prototype embodiment, data communication was provided by a USB communication protocol.

The prototype system was tested to determine the lag between a change in bioimpedance and changes in the output signals. This was achieved by connecting a computer-controlled potentiometer between electrodes 17A (see FIG. 3). The potentiometer was a digital potentiometer (AD5206, Analog Devices Norwood, Mass., USA) controlled by a microcontroller programmed to output a pre-defined waveform. The microcontroller was programmed to control the digital potentiometer to have a resistance that changed with time according to an asymmetric square wave of 2.5 kΩ amplitude. In a single period of 2.79 s the resistance was maintained at 5 kΩ but periodically decreased to 0 for approximately 11 ms and then restored to 5 kΩ. This pattern of resistance change created a short step function that allowed the monitoring of the delay between a change in bioimpedance and a change in the output respiration signal.

With the assumption that there is effectively no propagation delay between the change of the resistance and the output of the signal injecting amplifier, the propagation delay of the respiratory monitoring circuit can be characterized by comparing the output of the current-injecting amplifier and the output of the entire respiratory monitoring circuit. For experimental purposes the delay was defined by the time from the center of the lower resistance portion of the square wave to the lowest value of the detector circuit output. The measurement and signal processing lag from the prototype hardware design was determined to be 30 msec or roughly 1% of a standard respiratory cycle. As long as the processing performed for generating a gating of signal does not exceed the same order, the total lag will not exceed approximately 2-3% of the standard respiratory cycle. This lag is acceptable as, according to TG report 76, the total time delay of a real-time tracking/compensation system should be kept as short as possible and, in any case, not more than 0.5 seconds.

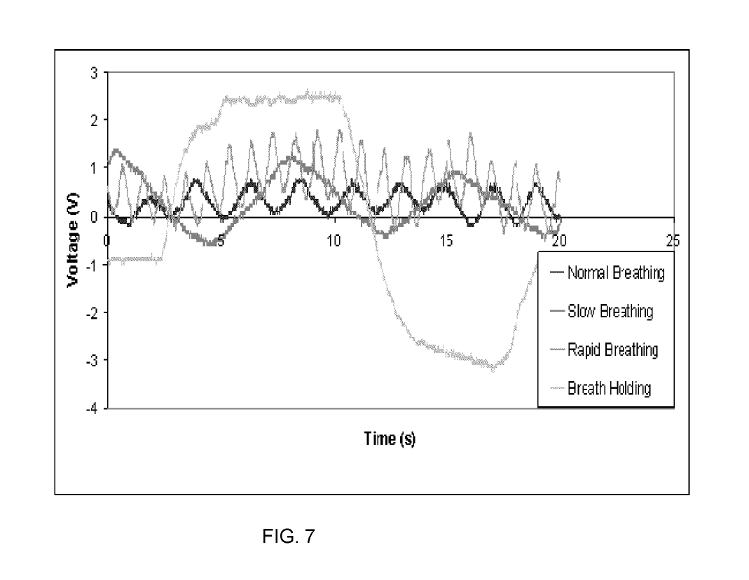

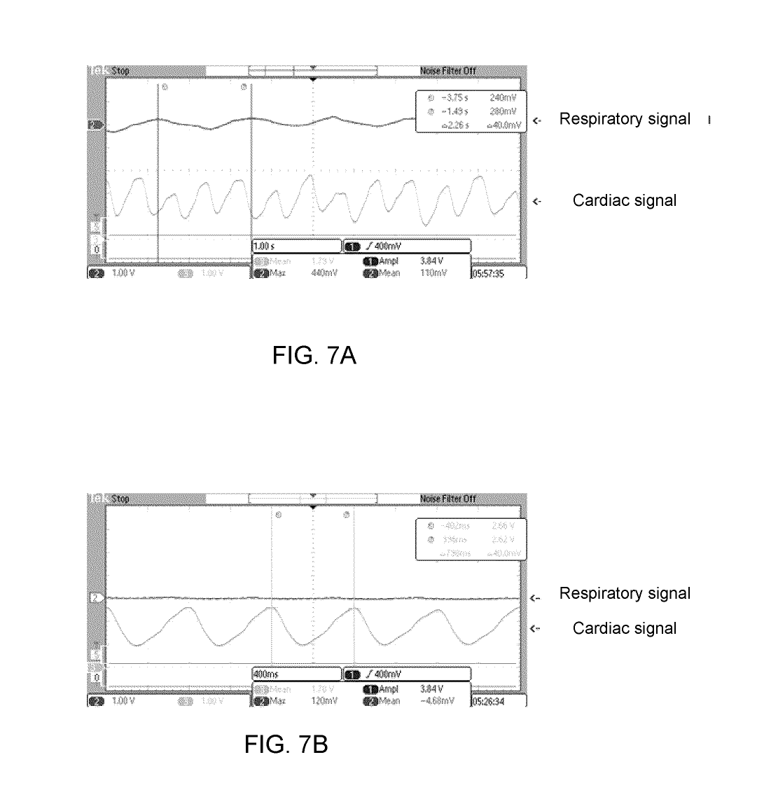

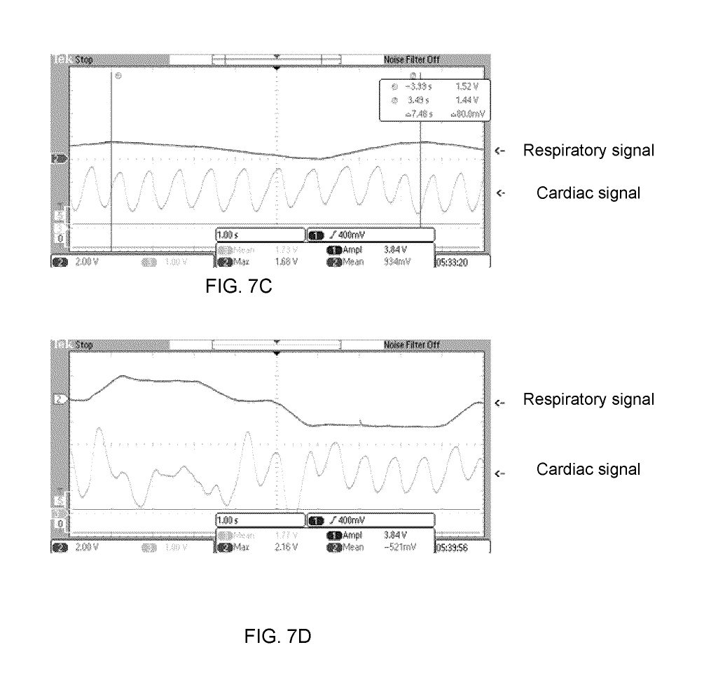

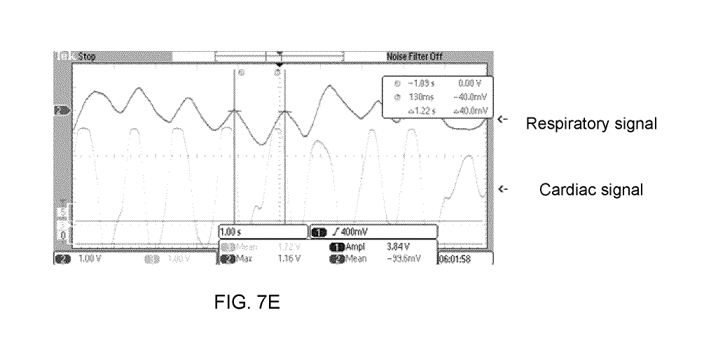

FIG. 7 demonstrates the ability of the prototype system to monitor respiration. Test subjects were instructed to intentionally control their breathing rate. FIG. 7 shows different breathing patterns recorded on a healthy volunteer exercising normal breathing, slow breathing, rapid breathing, inhaling, breath holding-exhaling, sequentially. The results show that the electrical impedance method could consistently trace the breathing pattern arbitrated by the test subject.

FIGS. 7A to 7E illustrate respiratory and cardiac signals obtained by a prototype bioimpedance monitor as described herein. It can be seen that the respiratory and cardiac signals change with different modes of breathing. FIG. 7A shows the case of normal breathing. It can be seen that during normal breathing, the respiratory rate had a period of approximately 2 seconds. The cardiac electrical signal was slightly distorted. FIG. 7B shows the case of breath holding. When the breath was held, the respiratory electrical signal remained at a steady level, and the cardiac electrical signal was undistorted with a period of approximately 0.8 seconds. FIG. 7C shows the case of long breathing. During a long breath, the respiratory signal showed a small fluctuation with a relatively long period. The cardiac signal was largely unaffected. FIG. 7D shows the case of deep breathing. In deep breathing the subject took a quick inhalation followed by a period of breath holding, and then air was exhaled followed by another period of breath holding. For a deep breath, the cardiac signal was most largely affected during the quick inhalation, the cardiac signal remained distorted when the lungs are filled with air. However, the cardiac signal returned to normal during the breath holding after the exhalation. FIG. 7E shows the case of rapid breathing. During a rapid inhalation/exhalation, the cardiac signal was largely influenced by the respiratory signal. It can be observed that the respiratory and cardiac signals have almost the same signal periods in FIG. 7E.

In some embodiments ECG signals are detected concurrently with bioimpedance signals. In some embodiments the ECG signals are detected using at least some of the same electrodes used for bioimpedance measurements. Such embodiments can acquire simultaneous respiratory, cardiac and ECG signals in real time using only 4 electrodes. These signals may be used for gating in radiotherapy and/or imaging. FIG. 3 shows an optional ECG signal detector 100. The output signal from amplifier 102 is filtered at highpass filter 102 and lowpass filter 104 to produce ECG signal 106. In some embodiments, highpass filter 102 has a cutoff frequency of 0.8 Hz. In some embodiments, lowpass filter has a cutoff frequency of 100 Hz.

In an example embodiment, ECG signals are acquired using the same electrode pair used to monitor bio-impedance changes due to cardiac motion (e.g. electrodes 17B). To obtain the ECG signal, a circuit is provided that detects and amplifies frequencies between 0.8 Hz and 100 Hz but suppresses signals outside this frequency region. The selection of such frequency band allows carrier signal 32A to be effectively excluded and the electrical potential generated by the sinoatrial node in the heart to be observed. Such electrical potential consists of mostly frequency components less than 100 Hz. Because of its myogenic nature, the ECG signal is more rhythmic and more immune to interference caused by the respiratory motion than the cardiac-bio-impedance signal. However, for gating purpose, it is the cardiac-induced impedance change that presents the actual contraction of the heart muscles. In some embodiments cardiac gating is performed using both the ECG and bioimpedance signals. For example, a gating signal that enables delivery of radiation may be delivered only when both the ECG signal and the cardiac bioimpedance signal satisfy specific criteria.

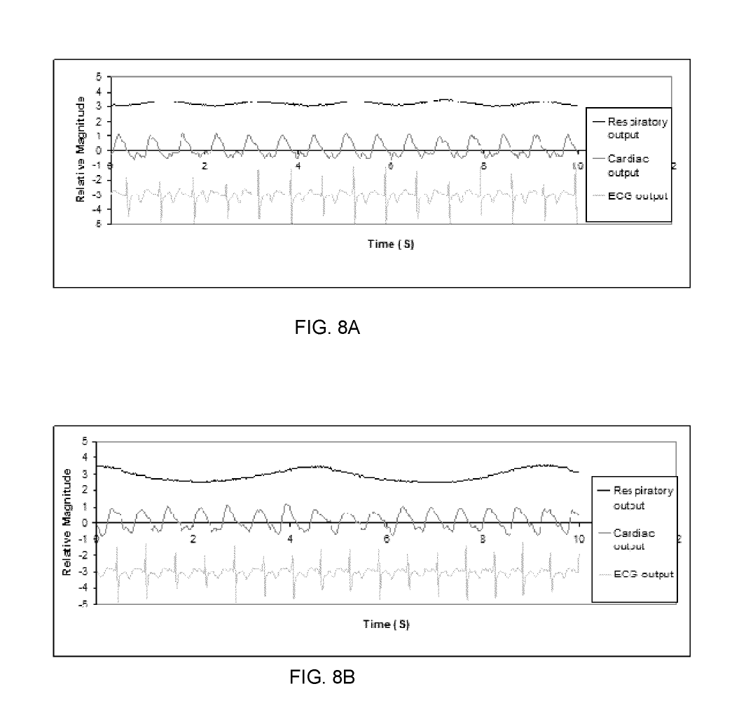

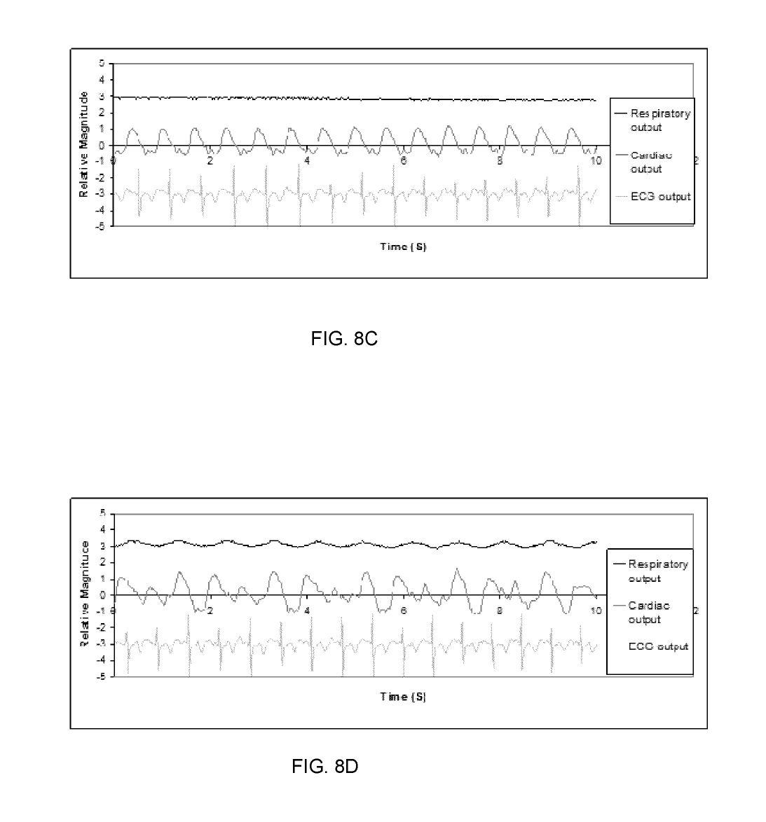

FIGS. 8A to 8D show ECG signals, bioimpedance-derived respiratory signals and bioimpedance-derived cardiac signals obtained for a 32 year-old male subject using four electrodes. FIG. 8A is for the case of normal-rate breathing. FIG. 8B is for the case of slow breathing. FIG. 8C is for the case of breath holding. FIG. 8D is for the case of rapid breathing.

Tests were conducted to verify functionality of a prototype system in a radiation environment. The prototype system was placed in the vicinity of a high-energy X-Ray beam. The effect of radiation on the system was tested using the variable resistance circuit mentioned above. The digital potentiometer was programmed to output a resistance that varied sinusoidally with a period of approximately 3 seconds to simulate breathing of a subject. A pair of lead wires embedded in a 50-cm coaxial cable connected the output terminals of the current sourcing amplifier across the variable resistance circuit. The lead wires were placed inside the radiation field and directly irradiated with a 6 MV beam to ˜100 cGy dose at 400 MU/min. The field size was 10×10 cm². The respiratory monitoring circuit and the variable resistance circuit were located outside the radiation field. The output of the respiratory monitoring circuit was recorded with and without the high-energy beam switched on. In the first trial, the variable resistance was left running according to the input sinusoidal waveform for 120 seconds without irradiation. In the second trial, the X-Ray beam was switched on 60 seconds after recording started and turned off when the total dose reached 100 cGy. Results of the two test conditions were compared. No significant effect on the functionality of the system was observed when it was tested in a radiation environment with the electrode lead wires directly exposed to high-energy X-Rays.

Apparatus and methods as described herein have a wide variety of applications. One application of particular value is in delivering radiation in stereotactic ablative radiotherapy (SABR) lung and esophageal treatments, where long-term toxicities are often seen when high doses are given and even small errors in patient positioning or motion tracking can result in substantial overdoses to central airway or vascular structures. Another example application is stereotactic body radiotherapy (SBRT) where margins are often small minimal and it is desirable to spare central structures from high doses of radiation as much as possible. Another example application is breast radiotherapy. Many breast radiotherapy patients, have previously received cardiotoxic chemotherapy. As large numbers of women are treated with adjuvant breast or chest wall radiation, even small reductions in cardiac dose may be extremely significant to survival at a population level. Such a system is of particular value in treating left-sided breast cancer patients, lung cancer patients and upper abdominal malignancy patients (e.g. patients having malignancies in the pancreas or liver).

An advantage of the use of bioimpedance for respiratory gating is that bioimpedance measurements can accurately track the respiratory cycle even in patients with compromised lung function who exhibit paradoxical diaphragm motion (both as a single structure and with respect to the ventral rib cage). Paradoxical diaphragm motion can occur, for example, in patients with emphysema. As the population of lung cancer patients presenting for radiotherapy contains many patients with compromised pulmonary function, concerns about the use of the diaphragm as a surrogate indicator of lung tumor motion are extremely relevant. The electrical impedance signal is not based merely on diaphragm motion, but rather depicts changes in lung volume and geometry which has a potential to accurately gate in situations of paradoxical chest wall or diaphragm motion.

In some alternative embodiments, phase of the cardiac cycle and phase of the respiratory cycle are detected using separate signals that are distinguishable from one another. for example, the signals may have different frequencies and/or may be encoded in different ways. These signals may be injected into the subject using the same or different electrodes. In cases where current is injected or withdrawn at electrodes near the heart care should be taken to avoid current densities near the heart that could have undesirable health consequences. For example, the current of any signal passing through any electrodes near the heart may be limited to have a sufficiently low value that no excessive current densities can arise. In an example embodiment, the subject's cardiac cycle is monitored using a signal passed between a first electrode that is more remote from the heart (e.g. on a side of the subject's thorax) and a second electrode located nearer to the subject's heart (e.g. on the subject's thorax). Bioimpedance of the subjects tissues may be measured between these two electrodes or between two other electrodes located near to the subject's heart or between the second electrode and another electrode located near to the subject's heart. A cardiac cycle signal may be derived from this measured bioimpedance.

### INTERPRETATION OF TERMS

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”)). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a gating device may implement methods as described herein by executing software instructions in a program memory accessible to the processors.

In some embodiments, certain aspects of the invention may be implemented in software. For greater clarity, “software” includes any instructions executed on a processor, and may include (but is not limited to) firmware, resident software, microcode, and the like. Both processing hardware and software may be centralized or distributed (or a combination thereof), in whole or in part, as known to those skilled in the art. For example, software and other modules may be accessible via local memory, via a network, via a browser or other application in a distributed computing context, or via other means suitable for the purposes described above.

Software aspects of the invention may also be provided in the form of a program product. The program product may comprise any non-transitory medium which carries a set of computer-readable instructions which, when executed by a data processor, cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

Where a component (e.g. a filter, electrode, amplifier, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

### REFERENCES

1. Barber, D. C. A review of image reconstruction techniques for electrical impedance tomography. Medical Physics, 1989; 16(2): 162-169.
2. Davidson, K. G., A. D. Bersten, T. E. Nicholas, P. R. Ravenscroft, and I. R. Doyle. Measurement of tidal volume by using transthoracic impedance variations in rats. Journal of Applied Physiology, 1999; 86(2): 759-766.
3. Faes, T. J. C., H. A. van der Meij, J. C. de Munck, and R. M. Heethaar. The electric resistivity of human tissues (100 Hz-10 MHz): a meta-analysis of review studies. Physiology, 1999; 20(4): R1.
4. Koivumaki T., M. Vauhkonen, J. T. Kuikka, and M. A. Hakulinen. Bioimpedance-based measurement method for simultaneous acquisition of respiratory and cardiac gating signals. Physiological Measurement, 2012; 33: 1323-1334.
5. Marinova I. and V. Mateev. Determination of electromagnetic properties of human tissue. World Academy of Science, Engineering and Technology 42, 2010.
6. Saw, C. B., E. Brandner, R. Selvaraj, H. Chen, M. Saiful Huq, and D. E. Heron. A review on the clinical implementation of respiratory-gated radiation therapy. Biomedical Imaging and Intervention Journal, 2007; 3(1): e40.
7. Visser, K. R. Electric properties of flowing blood and impedance cardiography. Annals of Biomedical Engineering, 1989; 17: 463-473.

1. Apparatus for gating delivery of radiation to a subject, the apparatus comprising:
a signal generator having first and second outputs respectively connectable to first and second electrodes, the signal generator operative to apply an electrical sensing signal between the first and second electrodes;
a first monitoring circuit configured to monitor characteristics of the electrical sensing signal to yield a first output signal representative of an electrical impedance between the first and second electrodes;
a second monitoring circuit having first and second inputs connectable to third and fourth electrodes and configured to monitor an electrical potential between the third and fourth electrodes to yield a second output signal, the second monitoring circuit comprising an analog filter having a bandpass filter characteristic with a passband including frequencies in the range of 1-2 Hz; and
a gating circuit connected to process the first and second output signals to yield a gating signal.

2. Apparatus according to claim 1 wherein the electrical sensing signal has a frequency exceeding 1 kHz and the first and second monitoring circuits each comprise an analog filter tuned to pass the frequency of the electrical sensing signal.

3. Apparatus according to claim 1 wherein the first monitoring circuit comprises a first signal amplitude detector and a first difference circuit connected to subtract a first DC offset from an output of the first signal amplitude detector upstream from the first analog filter.

4. (canceled)

5. Apparatus according to claim 3 comprising a control circuit connected to control a magnitude of the subtracted DC offset, wherein the difference circuit comprises a difference amplifier and the control circuit comprises a digital to analog converter having an output connected to set a voltage applied to one input of the difference amplifier.

6. Apparatus according to claim 3 comprising a control circuit connected to control a magnitude of the subtracted DC offset, wherein the control circuit comprises a programmable processor configured by software to monitor a DC component of a signal output by the difference amplifier and to dynamically vary the set voltage to reduce the DC component of the signal output by the difference amplifier to be below a threshold.

7. Apparatus according to claim 1 wherein the second monitoring circuit comprises a second signal amplitude detector and a second difference circuit connected to subtract a second DC offset from an output of the second signal amplitude detector upstream from the second analog filter; wherein the first monitoring circuit comprises an analog filter having a low pass or bandpass filter characteristic downstream from the second difference circuit.

8. (canceled)

9. Apparatus according to claim 3 wherein the gating circuit is configured to monitor a rate of change of a DC component of the output of the first signal amplitude detector and to set the gating signal to inhibit radiation delivery if the rate of change meets or exceeds a threshold.

10. Apparatus according to claim 3 wherein the gating circuit is configured to monitor a difference between a DC component of the output of the first signal amplitude detector at a first time and a present time and to set the gating signal to inhibit radiation delivery if the difference meets or exceeds a threshold.

11. Apparatus according to claim 1 wherein the gating circuit is configured to monitor a phase of the first output signal and a phase of the second output signal and to set the gating signal to inhibit radiation delivery unless the phase of the first output signal and the phase of the second output signal each satisfy a predetermined criterion.

12. Apparatus according to claim 1 wherein the gating circuit is configured to monitor an amplitude, frequency or amplitude and frequency of an AC component of the first output signal and to set the gating signal to inhibit radiation delivery based at least in part on values of the amplitude, frequency or amplitude and frequency of the AC component.

13. Apparatus according to claim 3 wherein the gating circuit is configured to periodically sample a DC component of the output of the first signal amplitude detector and to set the gating signal to inhibit delivery of radiation if more than a threshold number of the samples in a current time window deviate from a predefined range; wherein the predefined range is a range around an average or median value of the samples.

14. (canceled)

15. Apparatus according to claim 1 wherein the gating circuit is configured to set the gating signal to inhibit delivery of radiation if a rate of change of the frequency or amplitude of an AC component of the first output signal exceeds a threshold rate.

16. Apparatus according to claim 1 wherein the gating circuit is configured to set the gating signal to inhibit delivery of radiation if a frequency or amplitude of an AC component of the first output signal is outside of a predetermined range.

17. Apparatus according to claim 1 comprising a differentiating amplifier connected to output a rate of change of a frequency and/or amplitude of an AC component of the first output signal.

18. (canceled)

19. Apparatus according to claim 1 in combination with a radiotherapy delivery apparatus wherein the gating signal is connected to selectively enable and inhibit delivery of radiation by the radiotherapy delivery apparatus.

20-22. (canceled)

23. Apparatus according to claim 1 comprising an ECG circuit connected to process the potential difference at the inputs of the second monitoring circuit to yield an ECG output signal wherein the ECG circuit comprises a filter circuit configured to detect and amplify frequencies in the range of about 0.8 Hz to about 100 Hz and to suppress other frequencies.

24. Apparatus according to claim 1 comprising an ECG circuit connected to process the potential difference at the inputs of the second monitoring circuit to yield an ECG output signal wherein the gating circuit is connected to receive the ECG output signal and configured to generate the gating signal based in part on the ECG signal.

25. Apparatus according to claim 24 wherein the gating circuit is configured to inhibit delivery of radiation unless the ECG output signal and the second output signal each satisfy predetermined criteria.

26. Apparatus according to claim 1 wherein the signal generator comprises a controlled current source configured to maintain a preset safe current between the first and second electrodes and the first monitoring circuit monitors a potential difference between the first and second electrodes.

27. A method for generating a gating signal for gating delivery of radiation to a subject, the method comprising:
applying an electrical sensing signal between first and second electrodes in contact with a subject;
measuring an impedance between the first and second electrodes to produce an impedance signal;
measuring a voltage between third and fourth electrodes in contact with the subject to produce a voltage signal and processing the voltage signal to determine an amplitude of the voltage signal;
filtering the impedance signal in the analog domain to remove signal components with frequencies above a first threshold frequency to produce a first output signal;
filtering the processed voltage signal in the analog domain to remove signal components outside of a frequency band, the frequency band including frequencies in the range of 1-2 Hz, to produce a second output signal; and
processing the first and second output signals to generate a gating signal.

28. (canceled)

29. A method according to claim 27 comprising, before filtering the impedance signal:
measuring the amplitude of the impedance signal; and
subtracting a first DC offset from the amplitude of the impedance signal;
wherein the method further comprises adjusting the first DC offset to maintain the amplitude of the impedance signal below a threshold.

30. (canceled)

31. A method according to claim 27 comprising, before filtering the voltage signal, subtracting a second DC offset from the amplitude of the voltage signal.

32. A method according to claim 27 wherein processing the first and second output signals to generate a gating signal comprises:
monitoring a rate of change of a DC component of the impedance signal; and
generating a gating signal that inhibits radiation delivery if the rate of change meets or exceeds a threshold.

33. A method according to claim 27 wherein processing the first and second output signals to generate a gating signal comprises:
monitoring a difference between a DC component of the impedance signal at a first time and a present time; and
generating a gating signal that inhibits radiation delivery if the difference meets or exceeds a threshold.

34. A method according to claim 27 wherein processing the first and second output signals to generate a gating signal comprises:
monitoring a phase of the first output signal and a phase of the second output signal; and
generating a gating signal that inhibits radiation delivery unless the phase of the first output signal and the phase of the second output signal each satisfy a corresponding predetermined criterion.

35. A method according to claim 27 wherein processing the first and second output signals to generate a gating signal comprises:
monitoring an amplitude, frequency or amplitude and frequency of an AC component of the first output signal; and
generating a gating signal that inhibits radiation delivery based at least in part on values of the amplitude, frequency or amplitude and frequency of the AC component.

36. A method according to claim 27:
wherein processing the first and second output signals to generate a gating signal comprises:
periodically sampling a DC component of the impedance signal; and
generating a gating signal that inhibits radiation delivery if more than a threshold number of the samples in a current time window deviate from a predefined range; and
wherein the predefined range is a range around an average or median value of the samples.

37. (canceled)

38. A method according to claim 27 wherein processing the first and second output signals to generate a gating signal comprises:
generating a gating signal that inhibits radiation delivery if a rate of change of a frequency or amplitude of an AC component of the first output signal exceeds a threshold rate.

39. A method according to claim 27 wherein processing the first and second output signals to generate a gating signal comprises generating a gating signal that inhibits radiation delivery if a frequency or amplitude of an AC component of the first output signal goes outside a predetermined range.

40. A method according to claim 27 comprising sampling the first and second output signals and generating the gating signal based at least in part on the sampled first and second output signals.

41. (canceled)

42. A method according to claim 27 comprising processing the voltage signal to yield an ECG signal wherein processing the voltage signal to yield the ECG signal comprises amplifying frequencies in the range of about 0.8 Hz to about 100 Hz and suppressing other frequencies.

43. A method according to claim 27 comprising processing the voltage signal to yield an ECG signal and generating the gating signal based in part on the ECG output signal.

44. Apparatus for gating delivery of radiation to a subject, the apparatus comprising:
a first pair of electrodes for placing on either side of a subject's torso;
a second pair of electrodes for placing on the subject's torso in a vicinity of the subject's heart;
a first impedance-sensing circuit configured to monitor a first bioimpedance between the first pair of electrodes and to generate a respiration signal indicative of a phase of the subject's respiration cycle from the monitored first bioimpedance;
a second impedance-sensing circuit connected to monitor a potential difference between the second pair of electrodes and configured to monitor a second bioimpedance between the second pair of electrodes and to generate a cardiac signal indicative of a phase of the subject's cardiac cycle from the monitored second bioimpedance;
an ECG circuit configured to generate a ECG signal from the potential difference between the second pair of electrodes; and
a gating circuit connected to receive the cardiac signal and the respiration signal and configured to generate a gating signal based on at least the cardiac signal and the respiration signal.

45. Apparatus according to claim 44 wherein the gating circuit is configured to generate the gating signal based in part on the ECG signal.

46. Apparatus according to claim 44 wherein the first pair of electrodes are located along mid-axillary line on both the right and left sides of the subject's chest.

47. Apparatus according to claim 46 wherein one electrode of the second pair of electrodes is located at the level of the subject's xiphoid and a second electrode of the second pair of electrodes is located 2 cm lateral of the one electrode on the left side.

48. Apparatus for gating delivery of radiation to a subject, the apparatus comprising:
a first pair of electrodes for placing on either side of a subject's torso;
a second pair of electrodes for placing on the subject's torso in a vicinity of the subject's heart;
a first impedance-sensing circuit configured to monitor a first bioimpedance between the first pair of electrodes and to generate a respiration signal indicative of a phase of the subject's respiration cycle from the monitored first bioimpedance;
a second impedance-sensing circuit connected to monitor a potential difference between the second pair of electrodes and configured to monitor a second bioimpedance between the second pair of electrodes and to generate a cardiac signal indicative of a phase of the subject's cardiac cycle from the monitored second bioimpedance; and
a gating circuit connected to receive the cardiac signal and the respiration signal and configured to generate a gating signal based on at least the cardiac signal and the respiration signal;
wherein the first impedance sensing circuit is configured to subtract a DC offset from the monitored first bioimpedance and the gating circuit is connected to receive a signal indicative of a magnitude of the DC offset and to generate a gating signal based at least in part on the magnitude of the DC offset.

49-53. (canceled)

54. A method for creating a signal for gating delivery of radiation to a subject, the method comprising:
monitoring a first bioimpedance between the first pair of electrodes on either side of a subject's torso and generating a respiration signal indicative of a phase of the subject's respiration cycle from the monitored first bioimpedance;
monitoring a second bioimpedance between a second pair of electrodes on the subject's torso in a vicinity of the subject's heart based on a potential difference between the second pair of electrodes and generating a cardiac signal indicative of a phase of the subject's cardiac cycle from the monitored second bioimpedance;
subtracting a DC offset from the monitored first bioimpedance; and
generating a gating signal based at least in part on the magnitude of the DC offset.

55-56. (canceled)

57. A method according to claim 54 comprising monitoring a phase of the cardiac signal and a phase of the respiration signal and setting the gating signal to inhibit radiation delivery unless the phase of the cardiac signal and the phase of the respiration signal each satisfy a predetermined criterion.

58-61. (canceled)
